(19) United States
(12) Patent Application Publication
(10) Pub. No.: US 2025/0057536 A1
(43) Pub. Date: Feb. 20, 2025

(54) Electrolytically Detachable Intravascular Delivery Device

(71) Applicant: DePuy Synthes Products, Inc., Raynham, MA (US)

(72) Inventors: Ruijiao XU, Miami Lakes, FL (US); Jasia HOLLANDER, Miami, FL (US)

(21) Appl. No.: 18/451,725

(22) Filed: Aug. 17, 2023

(51) Int. Cl. A61B 17/12; A61L 31/02; A61L 31/10; A61L 31/14

(52) U.S. Cl. CPC A61B 17/12022; A61L 31/022; A61L 31/10; A61L 31/14; A61B 2017/00526; A61B 2017/00951; A61B 2017/12063

(57) ABSTRACT

An electrolytically detachable intravascular delivery system including a core wire made of a nickel-titanium-cobalt alloy; a distal coil disposed about the core wire; a distal electrical insulation covering of polyethylene terephthalate material adhered to the outer surface of the distal coil via a distal adhesive coating; a proximal coil made of a platinum alloy disposed about the core wire proximally of the distal coil; a proximal electrical insulation covering of polyethylene terephthalate material adhered to the outer surface of the proximal coil via a proximal adhesive coating. The core wire having an exposed section representing a detachment zone located in the axial direction between the proximal electrical insulating covering and the distal electrical insulation covering.

### BACKGROUND OF THE INVENTION

### Field of the Invention

The present disclosure relates to a device for intravascular delivery of an implantable medical device through the vasculature of the body to a target site. For instance, the disclosure is applicable to an intravascular device used to deliver an implantable medical device (e.g., an intrasaccular device for treatment of an aneurysm) and upon reaching the target site is electrolytically detachable.

### Description of Related Art

Implantable medical devices are widely used in various medical treatments and procedures, for example, treatment of an aneurysm. These conventional medical treatment procedures involve a device for intravascular delivery or navigation of an implantable medical device (e.g., intrasaccular device) to a target site (e.g., aneurysm) in the vessel to be treated. Upon reaching the target site, the implantable medical device is released or detached from the intravascular delivery device secured, connected or attached thereto. Conventional mechanisms or methods of detachment of the implantable medical device from the intravascular delivery and detachment device include: (i) mechanical detachment systems; or (ii) electrolytic detachment systems.

A mechanical detachment system is one in which the implantable medical device is secured to the delivery device via a core or securement wire releasable upon engagement of a mechanical mechanism. For example, pulling of the core wire in a proximal direction releases the securement wire from the implantable medical device allowing subsequent withdraw in a proximal direction the delivery device from the body while leaving in place within the vessel the implantable medical device properly positioned at the target site.

An electrolytic detachment system utilizes the body's electrolytes to dissolve at a detachment zone or region of the securement wire by which the implantable medical device is secured to the delivery device so that the two components are freed from each other. In conventional electrolytic detachment systems, with the exception of only the exposed detachment zone or region at the very distal end, the rest of the delivery device is electrically insulated with a polyimide material. Once the implantable medical device is navigated to the target site in the vessel using the delivery device, the interventionalist via activation of a button/switch on a detachment device initiates an electrical current that is transmitted through the securement wire of the delivery device (i.e., from the position at which the current is produced to the distal end/tip) and concentrates in the detachment zone or region devoid or free of electrical insulation (e.g., no polymer insulation). Within the exposed detachment zone or region, the generated electrical current initiates an electrolysis reaction dissolving the metal securement wire thereby releasing/severing/detaching/freeing the implantable medical device from the delivery device.

Conventional electrolytically detachable intravascular delivery devices employ a core or securement wire made of stainless steel insulated with polyimide (except for the detachment zone). The use of stainless steel insulated with polyimide poses several disadvantages. To maximize trackability while navigating through tortuous vasculature, it is desirable to minimize the outer diameter of the securement wire. Due to the strength characteristics of stainless steel minimizing the outer diameter (e.g., diameter ≤approximately 0.005″) to maximize trackability comes at the expense or disadvantage of increased risk of breakage. Another problem associated with using stainless steel as the securement or delivery wire is the time required to sever the wire and release/detach the implantable medical device. A conventional securement wire having a diameter greater than 0.005″ takes longer than approximately 2 minutes to detach the implantable medical device. Proper positioning of the implantable medical device at the target site is essential to successful treatment of the medical condition and hence any reduction/shortening in the time required to sever the securement wire is beneficial.

It is desirable to develop an improved electrolytically detachable intravascular delivery device in which the material used for the core/securement/delivery wire provides maximum trackability while navigating through the vasculature by minimizing the outer diameter (e.g., diameter ≤approximately 0.005″) of the securement wire without increased risk of potential breakage and also reducing the time (e.g., less than approximately 2 minutes) to sever in order to detach/release the implantable medical device.

### SUMMARY OF THE INVENTION

An aspect of the present disclosure is directed to an improved electrolytically detachable intravascular delivery device with optimized trackability while navigating through the vasculature to deliver the implantable medical device to the desired target site.

Another aspect of the present disclosure relates to an improved electrolytically detachable intravascular delivery device in which the outer diameter of the core/securement/delivery wire is minimized without increased risk of potential breakage.

Still another aspect of the present disclosure is directed to an improved electrolytically detachable intravascular delivery device in which the time to sever the core/securement/delivery wire thereby detaching/releasing the implantable medical device is minimized.

While still yet another aspect of the present disclosure relates to an improved electrolytically detachable intravascular delivery device in which the strength of the core/securement/delivery wire within the detachment zone (i.e., exposed section free from electrical insulation) is maximized.

Yet another aspect the present disclosure is directed to an improved electrolytically detachable intravascular delivery device in which the core wire is made of an alloy of Nickle-titanium and cobalt (NiTiCo) electrically insulated with an outer layer of polyethylene terephthalate (PET) material everywhere except along an exposed (devoid or free of electrical insulation) detachment zone region, wherein the surface and hence strength of the bond between the PET material and core wire is maximized by placing a distal coil disposed therebetween.

### BRIEF DESCRIPTION OF THE DRAWING

The foregoing and other features of the present disclosure will be more readily apparent from the following detailed description and drawings of illustrative of the disclosure wherein like reference numbers refer to similar elements throughout the several views and in which:

FIG. 1A is a longitudinal/axial cross-sectional view of the electrolytically detachable intravascular delivery device;

FIG. 1B is an enlarged longitudinal/axial cross-sectional view of section I(B) of the distal section of the electrolytically detachable intravascular delivery device of FIG. 1A;

FIG. 1C is a radial cross-sectional view along lines I(C)-I(C) of the electrolytically detachable intravascular delivery device of FIG. 1A;

FIG. 1D is a radial cross-sectional view along lines I(D)-I(D) of the electrolytically detachable intravascular delivery device of FIG. 1A;

FIG. 1E is a radial cross-sectional view along lines I(E)-I(E) of the electrolytically detachable intravascular delivery device of FIG. 1A;

FIG. 1F is a radial cross-sectional view along lines I(F)-I(F) of the electrolytically detachable intravascular delivery device of FIG. 1A;

FIG. 1G is a side view of the electrolytically detachable intravascular delivery device of FIG. 1A loaded/assembled with the implantable medical device prior to detachment;

FIG. 1H is a longitudinal/axial view along lines 1(H)-1(H) of the electrolytically detachable intravascular delivery device with the loaded/assembled implantable medical device prior to detachment of FIG. 1G;

FIG. 1I is an enlarged view of distal section I in FIG. 1H;

FIG. 1J is an enlarged view of section J in FIG. 1I;

FIG. 2A is a side view of the proximal coil of the electrolytically detachable intravascular delivery device of FIG. 1A;

FIG. 2B is a radial cross-sectional view along lines II(B)-II(B) of the proximal coil of FIG. 2A;

FIG. 3 is a side view of the core wire of the electrolytically detachable intravascular delivery device of FIG. 1A;

FIG. 4A is a side view of the distal coil of the electrolytically detachable intravascular delivery device of FIG. 1A;

FIG. 4B is a radial cross-sectional view along lines IV(B)-IV(B) of the distal insulation coil of FIG. 4A;

FIG. 5A is a side view of the proximal coil spacer of the electrolytically detachable intravascular delivery device of FIG. 1A;

FIG. 5B is a radial cross-sectional view along lines V(B)-V(B) of the proximal coil spacer of FIG. 5A; and

FIG. 6 is a side view an exemplary detacher device for receiving therein the proximal end of the electrolytically detachable intravascular delivery device with the loaded implantable medical device of FIG. 1G once the implantable medical device has been delivered/navigated to the target site in the vessel; the detacher device produces an electrical current that initiates an electrolysis reaction dissolving the metal securement wire along the detachment zone thereby releasing/severing/detaching/freeing the implantable medical device from the electrolytically detachable intravascular delivery device.

### DETAILED DESCRIPTION OF THE INVENTION

The terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist.

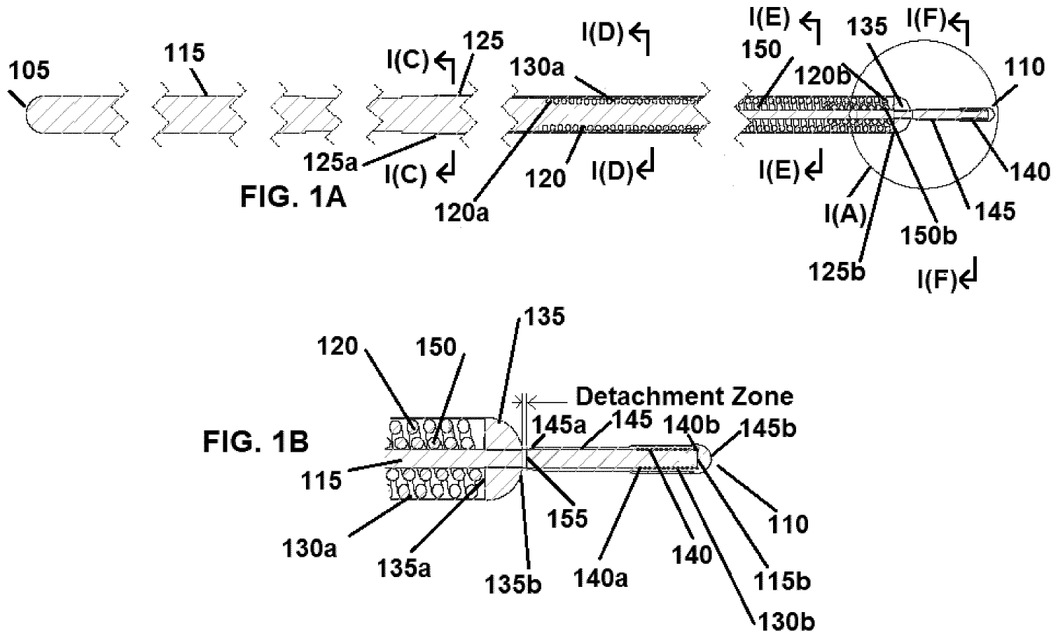

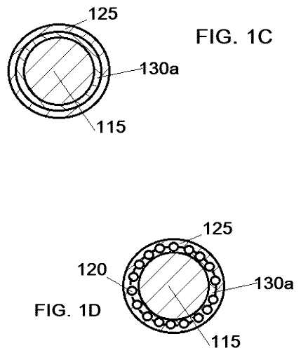

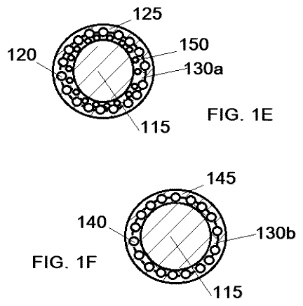

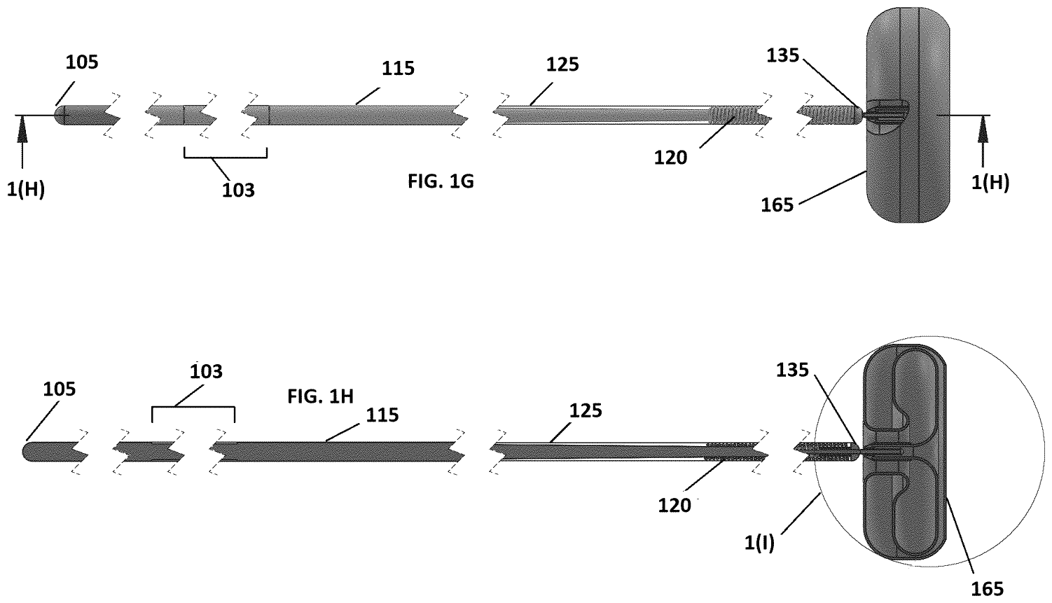

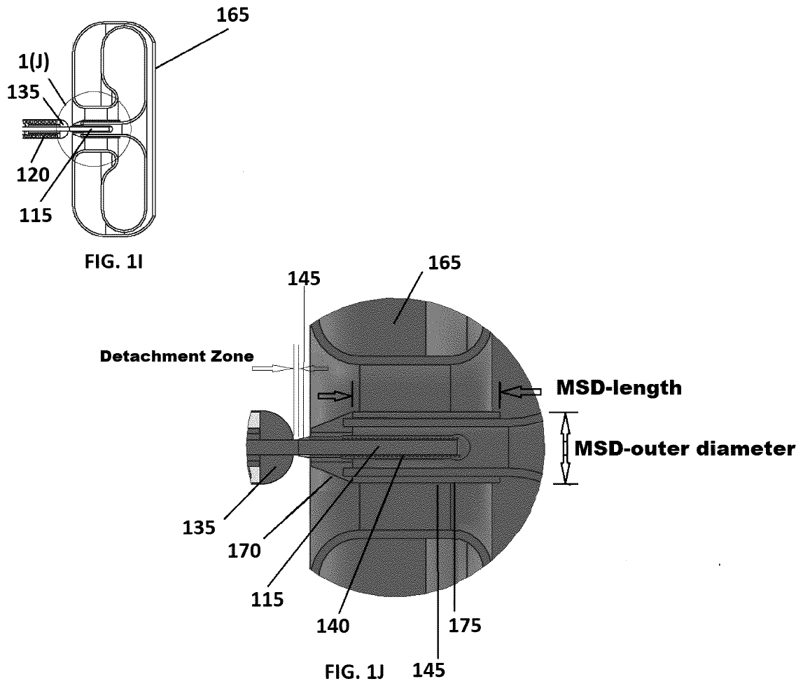

FIG. 1A is a longitudinal/axial cross-sectional view through the electrolytically detachable intravascular delivery device 100 in accordance with the present disclosure. Extending from the proximal end/tip 105 to the opposite distal end/tip 110 of the electrolytically detachable intravascular delivery device 100 is a core/securement/delivery wire 115 made of a nickel-titanium-cobalt alloy (NiTiCo; Nitinol and Cobalt) on a distal end/tip 115b of which is secured an implantable intravascular medical device 165 (e.g., an intrasaccular device for treatment of an aneurysm) (FIG. 1G). Use of a NiTiCo alloy for the core wire optimizes trackability and pushability during delivery of the implantable intravascular medical device 165 to the target site in the vessel and when properly positioned at the target site provides reliable and safe detachment of the metals in the blood stream during electrolytic detachment. Core wire 115 preferably has at least one, most preferably multiple, changes or transitions (e.g., step down) in outer diameter between the proximal end/tip 115a and the distal end/tip 115b of the core wire 115. Accordingly, the outer diameter of the core wire at the distal end/tip 115b is smaller than the outer diameter at the proximal end/tip 115a. Preferably, the final outer diameter at the distal end/tip 115b of the core wire 115 is approximately 0.004″. The one or more changes/transitions (e.g., step down) in outer diameter of the core wire 115 advantageously provides greater flexibility, trackability and deliverability while navigating through the tortuous pathway of the vasculature. However, at the sacrifice of flexibility, trackability and navigation through the vasculature, the present intravascular delivery and detachment system may use a core wire whose outer diameter at the distal end/tip 115b and opposite proximal end/tip 115a are equal.

In conventional electrolytically detachable intravascular delivery devices with the exception of the detachment zone, the remaining portions (both proximally and distally of the detachment zone) of the stainless-steel core wire are electrically insulated with polyimide which unfortunately has been identified under European Union regulations as a material imposing safety risks. Accordingly, with the NiTiCo core wire in accordance with the example of the present disclosure it is desirable to employ an electrical insulation material that avoids these safety risks. Instead of using polyimide, the NiTiCo core wire 115 is preferably electrically insulated with a polyethylene terephthalate (PET) material. However, the properties and characteristics associated with PET results in inferior, insufficient or reduced bond strength causing undesirable slippage of the melted distal electrical insulation covering from the distal end of the core wire. Specifically, the bond strength between the core wire and melted PET electrical insulation covering ranging between approximately 50 gf-approximately 100 gf is significantly less than the bond strength required to prevent premature detachment while navigating through tortuous vasculature (e.g., a bond strength of approximately 305 gf is needed for a braided intrasaccular device). To overcome this shortfall, the design of the present electrolytically detachable intravascular delivery device maximizes the bond surface area and hence the strength of the bond between the core wire 115 and the distal electrical insulation covering 145 (e.g., PET) by including a distal coil 140 disposed therebetween. When melted the distal electrical insulation covering 145 (e.g., PET) seeps between the winds/loops/turns of the distal coil 140 increasing the bond surface and thus significantly increasing the bond strength between the distal electrical insulation covering 145 and the core wire 115 minimizing the risk of slippage during delivery.

Referring to FIG. 1A, the distal coil 140 is disposed about the outer surface of a distal section of the core wire 115 (preferably having a smaller outer diameter relative to the larger outer diameter of the proximal end 115a of the core wire 115). Distal coil 140 is preferably made of tungsten, having a proximal end 140a and an opposite distal end 140b. The distal end 140b of the distal coil 140 is preferably flush with the distal end/tip 115b of the coil wire 115 such that no portion of the distal coil 140 extends in a distal direction beyond the distal end/tip 115b of the core wire 115 to prevent the distal end of the core wire from poking through the distal electrical insulation 145. Distal coil 140 is fully covered or enclosed by the distal electrical insulation covering 145, preferably heat shrink polyethylene terephthalate (PET), having a proximal end 145a and an opposite distal end 145b. The distal electrical insulation covering 145 is adhered to the outer surface of the distal coil 140 by a biocompatible adhesive coating 130b (e.g., Dymax MD® Medical Device Adhesive 1184-M-T). When melted the distal electrical insulation covering 145 (e.g., heat shrink PET) and adhesive coating 130b seeps between the individual winds/loops/turns of the distal coil 140 increasing the bond surface thereby significantly increasing the bond strength between the distal electrical insulation covering 145 and the core wire 115. Proximal end 145a of the distal electrical insulation covering 145 extends in the longitudinal/axial direction proximally of the proximal end 140a of the distal coil 140 to allow for a braid or marker band 155 to be physically secured (e.g., crimped) about the distal electrical insulation covering 145 proximally of the distal coil 140. While the opposite distal end 145b of the distal electrical insulation covering 145 preferably extends longitudinally/axially in a distal direction beyond the distal ends/tips 115b, 140b of the respective core wire 115 and the distal coil 140 (flush with one another) thus creating an atraumatic/blunt distal end 110. During delivery of the implantable medical device to the target site, the atraumatic distal end 110 advantageously prevents the distal end/tip 115b of the core wire 115 from poking through while also preventing injury to the surrounding tissue.

To further increase bond strength and minimize slippage, the braid or marker band 155 arranged proximally of the distal coil 140 is physically secured (e.g., crimped) around the proximal end/tip 145a of the distal electrical insulation covering 145. Arrangement of the distal coil 140 creates a mechanical lock by maximizing the separation strength between the marker band 155 and distal electrical insulation covering 145 secured beneath.

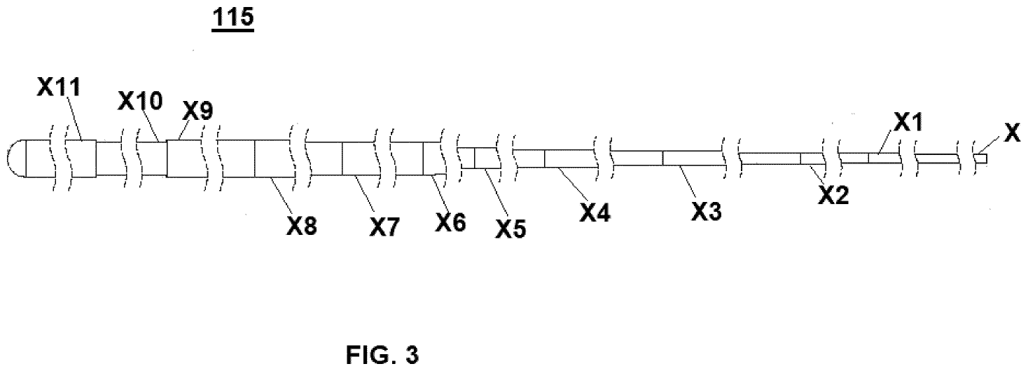

Disposed proximally of the distal coil 140 is a proximal coil 120, preferably formed using a wire made of a metal alloy including platinum either alone or with another metal (e.g., 92% platinum, 8% Tungsten). In comparison to that of stainless steel, the metal alloy including platinum selected for the proximal coil exhibits a higher yield point (i.e., point on a stress-strain curve representing the limit of elastic behavior (non-permanent or reversible deformation) at which the material begins to deform plastically (permanent or non-reversible deformation)). Accordingly, use of the proximal coil 120 made from a metal alloy including platinum exhibits greater flexibility or trackability while the electrolytically detachable intravascular delivery device navigates through the vasculature relative to that of stainless steel. Proximal coil 120 has a proximal end 120a, an opposite distal end 120b, a uniform/constant outer diameter and a uniform/constant inner diameter (i.e., inner passageway). The proximal coil 120 is positioned 360° about the outer surface of the core wire 115. By way of illustrative example, for the core wire 115 depicted in FIG. 3 the proximal coil 120 preferably extends in an axial/longitudinal direction with its proximal 120a end substantially aligned with a transition interface between adjacent sections having the largest transition or change (e.g., step down) in outer diameter (e.g., interface of sections X6 & X7), while its opposite distal end 120b is substantially aligned with a transition interface (i.e., proximal edge) of the section of the core wire having the smallest outer diameter (e.g., section X in FIG. 3). At the distal most change or transition (e.g., step down) in outer diameter of the core wire 115 (e.g., interface of sections X & X1 in the example coil wire 115 depicted in FIG. 3) a radial gap or space exists between the outer surface of the distal most section of the core wire 115 having an outer diameter smaller than that of the outer diameter of any section of the core wire 115 proximal thereto and the inner surface of inner passageway of the proximal coil 120. It would be complex during manufacture and assembly of the electrolytically detachable intravascular delivery device 100 to fill this radial gap or space with adhesive. In lieu of filling with an adhesive, the electrolytically detachable intravascular delivery device is preferably assembled with a mechanical spacer device (e.g., spacer coil) 150 disposed between the distal most section of the core wire 115 (e.g., X section in FIG. 3) having the smallest outer diameter and the inner surface of the proximal coil 120 filling the radial gap/space therebetween while simultaneously radially centering the core wire 115 within the inner passageway of the proximal coil 120. Spacer coil 150, preferably a stainless-steel wire, is permanently secured, e.g., indium soldered 160, within the inner passageway of the proximal coil 120.

Proximal electrical insulation covering 125, preferably a heat shrink PET, completely covers the outer surface of the entire proximal coil 120 from its proximal end 120a to its opposite distal end 120b with the core wire 115 extending through the inner passageway thereof. No portion of the proximal coil 120 is exposed (i.e., free of the proximal insulation covering 125). An adhesive coating 130a, such as Dymax MDR Medical Device Adhesive 1184-M-T, is used to secure the PET proximal electrical insulation covering 125 to the proximal coil 120. For optimum performance, the proximal electrical insulation covering 125 and the distal electrical insulation covering 145 are preferably both PET materials but need not necessarily be the same PET material nor have identical dimensions.

When melted the proximal electrical insulation covering 125 (e.g., heat shrink PET) and adhesive coating 130a seeps between the individual winds/loops/turns of the proximal coil 120 increasing the bond surface thereby significantly increasing the bond strength between the proximal electrical insulation covering 125 and the core wire 115. Preferably, the proximal end 125a of the proximal electrical insulation covering 125 extends axially/longitudinally in a proximal direction beyond the proximal end 120a of the proximal coil 120, while the distal end 125b of the proximal electrical insulation covering 125 extends in a distal direction beyond the distal end 120b of the proximal coil 120. For illustrative purposes only, FIG. 1A depicts the entire proximal coil 120 (from its proximal end 120a to its opposite distal end 120b) covered by adhesive 130a and solder 160, but less than full coverage of all coils is also contemplated. The length in an axial/longitudinal direction of the solder and/or adhesive joints for each loop or coil of the proximal coil 120 is preferably approximately 1 mm-approximately 2 mm.

The distal end 125b of the proximal electrical insulation covering 125 and the distal end 120b of the proximal coil 120 together are sealed off by a glue joint 135 having a blunt or atraumatic contour (e.g., domed, hemispherical or concave). For instance, the glue joint 135 may be a biocompatible ultraviolet cured adhesive including such chemicals as acrylate, ester, methacrylate ester monometer, or acrylic acid. Distal end/tip 125b of the proximal electrical insulation covering 125 abuts the proximal end 135a of the glue joint 135 without any gap therebetween in the axial/longitudinal direction. The extent of coverage of the proximal coil 120 with the adhesive coating 130a may vary, as desired, covering the entire proximal coil 120 continuously from its proximal end 120a to its distal end 120b (as illustrated in FIG. 1A) or only discrete portions (e.g., discontinuous with gap(s)/space(s) free from adhesive therebetween). To establish a strong bond, preferably a portion of the distal end/tip 120b of the proximal coil 120 extends in the longitudinal/axial direction into so as to be secured within the glue cap or joint 135 that is free from solder.

Proximal end 145a of the distal electrical insulation covering 145 is separated a predetermined distance in a longitudinal/axial direction from the distal end 135b of the glue joint 135 defining therebetween the detachment zone or region (DZ) in which the core wire 115 is exposed (i.e., free of any covering, for example, electrical insulation covering, adhesive or solder). Within the detachment zone, the core wire 115 is free of undercutting or necking (i.e., reducing of outer diameter). Minimal tensile break force on core wire 115 in the detachment zone is preferably approximately 800 gf.

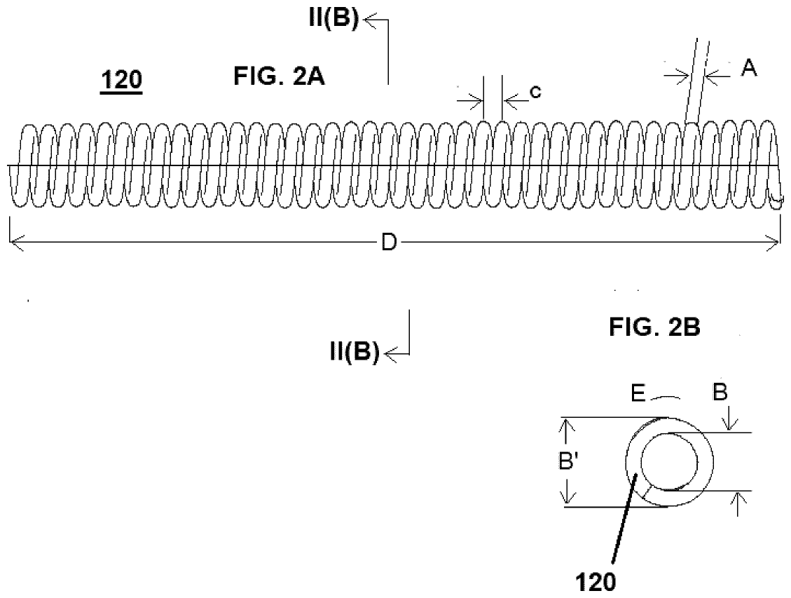

FIG. 2A is a side view of the proximal coil 120 of FIG. 1A, while FIG. 2B represents a radial cross-sectional view along lines II(B)-II(B) of FIG. 2A. Preferably, proximal coil 120 is manufactured from a single continuous wire made of a metal alloy including platinum alone or with other metals (e.g., 92% platinum and 8% tungsten), with the wire having a diameter (A) of approximately 0.003″±0.0002″. The single continuous wire in the illustrated example wound in a right-hand winding direction (E) into a coil or spiral shape (e.g., about a mandrel) has an outer diameter preferably in a range between approximately 0.0165″-approximately 0.0020″ to approximately 0.0165″±0.0010″, an inner diameter (B) preferably of approximately 0.010″, a pitch (C) of approximately 0.0032″±approximately 0.0003″, and a preferred length (D) in the axial/longitudinal direction of approximately 13.031″±0.080″.

FIG. 3 depicts a side view of an example of the core wire 115 of FIG. 1A made of NiTiCo with a chemical etching surface (CES) finish on its distal section of length in the axial/longitudinal direction of preferably approximately 5.0″±approximately 0.1″. The proximal end 115a of the core wire 115 is preferably rounded smooth and portions of the oxide layer are removed from the wire outer surface. The example core wire 115 in FIG. 3 comprises a plurality of segments (X, X1, X2, X3, X4, X5, X6, X7, X8, X9, X10, X11) one immediately following the other with some segments being tapered and those remaining segments being uniform (i.e., straight) in outer diameter. In a preferred example, starting from the distal end 115b and advancing towards the opposite proximal end 115a:
a distal most segment X preferably has an axial/longitudinal length≥approximately 0.25″ from a proximal end/tip of segment X having an outer diameter of approximately 0.0040″±approximately 0.0004″ to its distal most end/tip;
segment X1 tapers approximately 0.300″±approximately 0.045″ within an axial/longitudinal length from a proximal end/tip of segment X1 having an outer diameter of approximately 0.0045″±approximately 0.004″ to a proximal end/tip of segment X with an outer diameter of approximately 0.0040″±approximately 0.0004″ at the distal end/tip of segment X1;
segment X2 has a taper of approximately 2.00 within an axial/longitudinal length of approximately 2.300″±approximately 0.100″ (from a proximal end/tip of segment X2 to a proximal end/tip of segment X) with an outer diameter of approximately 0.0045″±approximately 0.0004″ at the distal end/tip of segment X2;
segment X3 has a taper of approximately 6.00 within an axial/longitudinal length of approximately 8.300″±approximately 0.100″ (from a proximal end/tip of segment X3 to a proximal end/tip of segment X) with an outer diameter of approximately 0.0055″±approximately 0.0004″ at the distal end/tip of segment X3;
segment X4 has a taper of approximately 4.00 within an axial/longitudinal length of approximately 12.300″±approximately 0.100″ (from a proximal end/tip of segment X4 to a proximal end/tip of segment X) with an outer diameter of approximately 0.0065″±approximately 0.0004″ at the distal end/tip of segment X4;
segment X5 has a taper of approximately 0.70 within an axial/longitudinal length of approximately 13.000″±approximately 0.100″ (from a proximal end/tip of segment X5 to a proximal end/tip of segment X) with an outer diameter of approximately 0.0085″±approximately 0.0004″ at the distal end/tip of segment X5;
segment X6 has a taper of approximately 0.25 within an axial/longitudinal length of approximately 13.250″±approximately 0.100″ (from a proximal end/tip of segment X6 to a proximal end/tip of segment X) with an outer diameter of approximately 0.010″±approximately 0.0004″ at the distal end/tip of segment X6;
segment X7 no taper (i.e., straight) within an axial/longitudinal length of approximately 17.800″±approximately 0.100″ (from a proximal end/tip of segment X7 to a proximal end/tip of segment X) with an outer diameter of approximately 0.0155″±approximately 0.0004″ at the distal end/tip of segment X7;
segment X8 has a taper of approximately 0.50 within an axial/longitudinal length of approximately 18.300″±approximately 0.100″ (from a proximal end/tip of segment X8 to a proximal end/tip of segment X) with an outer diameter of approximately 0.0155″±approximately 0.0004″ at the distal end/tip of segment X8 (unchanged from that of segment X7);
segment X9 no taper (i.e., straight) within an axial/longitudinal length of approximately 150 cm±approximately 1 cm (from a proximal end/tip of segment X9 to a proximal end/tip of segment X) with an outer diameter of approximately 0.0180″±approximately 0.0005″ at the distal end/tip of segment X9;
segment X10 no taper (i.e., straight) within an axial/longitudinal length of approximately 2.95 cm±approximately 0.25 cm (from a proximal end/tip of segment X10 to a proximal end/tip of segment X9) with an outer diameter≥approximately 0.0165; {Segment X10 of the core wire in the example illustrated in FIG. 3 corresponding to section 103 in FIG. 1A is ground down to a reduced diameter so that the segment is shiny serving as a visible with the naked eye fluoro saver/safety marker on a proximal section of the core wire indicating when the implantable medical device on the distal end is approaching the distal end of the delivery catheter (e.g., microcatheter) before turning on the fluoroscopic imaging to minimize radiation exposure.}
segment X11 no taper (i.e., straight) having an outer diameter of 0.0180″±0.0005″ within an axial/longitudinal length≥approximately 78.74 cm (from a proximal end/tip of segment X11 to a proximal end/tip of segment X).

Proximal electrical insulation covering (e.g., heat shrink PET) 125 preferably has a length in the axial/longitudinal direction≥approximately 50.0″, an expanded wall radial thickness of approximately 0.00050″±approximately 0.00010″, and an expanded inner diameter of approximately 0.024″±approximately 0.001″. Whereas, distal electrical insulation covering (e.g., heat shrink PET) 145 preferably has an expanded wall radial thickness of approximately 0.00040″±approximately 0.00010″ and an expanded inner diameter of approximately 0.007″±approximately 0.001″.

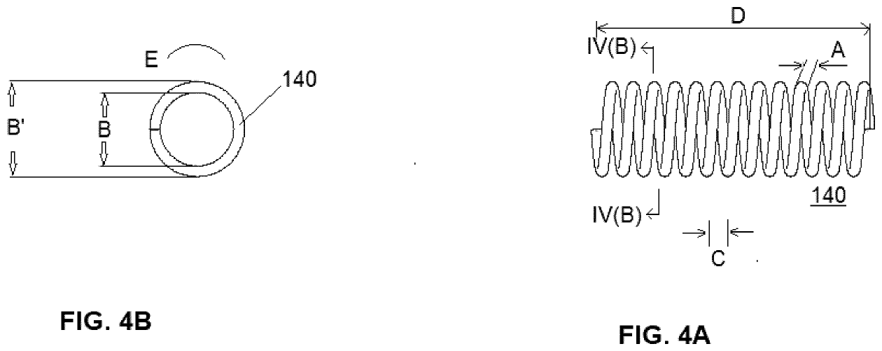

FIG. 4A is a side view of an example of the distal coil 140 in FIG. 1A, while FIG. 4B represents a radial cross-sectional view along lines IV(B)-IV(B) of FIG. 4A. Preferably, distal coil 140 is manufactured from a single continuous wire made of tungsten, with the wire having a diameter (A) of approximately 0.001″±approximately 0.0003″. The single continuous wire in the example in FIGS. 4A & 4B wound in a right-hand winding direction (E) into a coil or spiral shape (e.g., about a mandrel) has an outer diameter (B′) preferably of approximately 0.0064″±approximately 0.0003″, an inner diameter (B) preferably of approximately 0.004″, a pitch (C) of approximately 0.0015″±approximately 0.0003″, and a preferred length (D) in the axial/longitudinal direction of approximately 0.017″±0.003″.

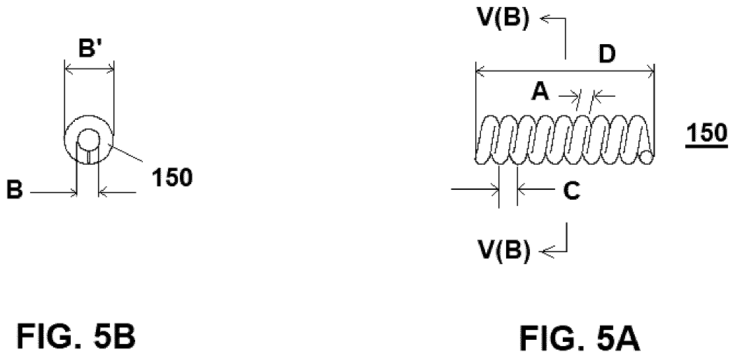

An example of the spacer coil 150 of FIG. 1A is shown alone in FIG. 5A, while FIG. 5B is a radial cross-sectional view along lines V(B)-V(B) of the spacer coil in FIG. 5A. Preferably, spacer coil 150 is manufactured from a single continuous wire made of stainless steel (e.g., 304V stainless steel) having a diameter (A) of approximately 0.0025″±approximately 0.0003″. The example single continuous wire spacer coil 150 in FIGS. 5A & 5B wound in a right-hand winding direction (E) into a coil or spiral shape (e.g., about a mandrel) has an outer diameter (B′) preferably of approximately 0.0094″±approximately 0.0003″, an inner diameter (B) preferably of approximately 0.0044″, a constant pitch (C) of approximately 0.0035″±approximately 0.0003″, and a preferred length (D) in the axial/longitudinal direction of approximately 0.030″±0.010″.

By way of illustrative example, the longitudinal/axial length of the proximal electrical insulation covering 125 is preferably greater than or equal to approximately 50″ from its proximal end/tip 125a to the opposite distal end/tip 125b that coincides/abuts the proximal end/tip 135a of the glue joint 135. An axial/longitudinal length from the proximal end 120a of the proximal coil 120 to distal end 135b of the glue joint 135 is preferably ≤ approximately 0.0190″. Solder and glue joints between each turn/wind of the proximal coil 120 with that of the outer surface of the spacer coil 150 or the proximal electrical insulation covering (e.g., heat shrink PET) 125 is preferably approximately 1 mm-approximately 2 mm in the longitudinal/axial direction. There is no solder distal of the distal end 120b of the proximal coil 120 within the region of the glue joint 135. The step down in outer diameter of the proximal coil 120 and outer diameter of the core wire 115 is preferably ≥approximately 0.0135″. At the distal end of the proximal coil 120 glue joint 135 preferably has a length in the axial/longitudinal direction from its proximal end 135a to the opposite distal most end 135b of approximately 0.010″±approximately 0.005″. An axial/longitudinal length of the detachment zone or region (i.e., exposed section of coil wire 115 free from electrical insulation covering (125, 145)) from the distal end 135b of the glue joint 135 to the proximal end 145a of the distal electrical insulation covering 145 is preferably approximately 0.0025″±approximately 0.0015″. At the proximal end 145a of the distal electrical insulation covering 145 the outer diameter is preferably approximately 0.0040″±approximately 0.0004″. Continuing in this exemplary preferred example, an axial/longitudinal length from the proximal end/tip 145a to the distal end/tip 145b of the distal electrical insulation covering 145 is preferably approximately 0.060″±approximately 0.005″, while the axial/longitudinal length from the distal end 115b of the core wire 115 to the distal end/tip 145b of the distal electrical insulation covering 145 is approximately 0.0030″±approximately 0.0055″. A maximum outer diameter of the distal electrical insulation covering 145 is ≤approximately 0.0095″. An axial/longitudinal length extending from the proximal end 140a of the distal coil 140 to the distal most end 145b of the distal electrical insulation covering 145 is preferably ≤approximately 0.025″. Joint minimal tensile break force between the proximal coil 120, spacer coil 150 and core wire 115 is preferably approximately 350.0 gf (approximately 0.77 lbf). While joint minimal tensile break force between distal coil 140, core wire 115 and distal electrical insulation covering (e.g., PET) 145 is preferably approximately 350.0 gf (approximately 0.77 lbf). When manufacturing the proximal coil 120 the outer diameter of each turn/wind/loop is preferably substantially equal. The solder 160 used is preferably a surgical grade alloy that flows at a temperature of approximately 430° F. such as 96.5% Sn, 3.5% Ag (Lead-free & Cadmium-free). As previously mentioned, the proximal electrical insulation covering (e.g., heat shrink PET) 125 and distal electrical insulation covering (e.g., heat shrink PET) 145 may be same of different materials and have the same or different dimensions.

FIGS. 1G-1J depict various views of the assembled electrolytically detachable intravascular delivery device 100 of FIG. 1A with an implantable medical device (e.g., the Medusa Multi-Coil® manufactured by Endoshape, Inc.) 165 loaded thereon. During manufacture spacer coil 150 is concentrically assembled within the inner passageway of the proximal coil 120 and permanently secured (e.g., soldered) together. The assembled components are then threaded onto (slid over) without flexing the core wire 115 radially centered therein. For the example core wire 115 depicted in FIG. 3, proximal coil 120 is arranged to extend in an axial/longitudinal direction with its proximal 120a end substantially aligned with the interface between adjacent sections having the largest transition or change (e.g., step down) in outer diameter (e.g., interface of sections X6 & X7), while its opposite distal end 120b is substantially aligned with an interface (i.e., proximal edge) of the distal most section of the core wire having the smallest outer diameter (e.g., section X in FIG. 3).

Next, the adhesive coating 130a is applied to the outer surface of the proximal coil 120, before the proximal electrical insulation covering 125 (e.g., heat shrink PET) is loaded onto the proximal coil 120 adhering the two components together. When melted, the proximal electrical insulation covering 125 (e.g., heat shrink PET) and adhesive coating 130a seeps between the individual winds/loops/turns of the proximal coil 120 thereby increasing the bond surface and hence bond strength between the proximal electrical insulation covering 125 (e.g., heat shrink PET) and the coil wire 115. Thereafter, the glue joint 135 is created to secure the distal end 120b of the proximal coil 120 and distal end 150b of the spacer coil 150 to the outer surface of the core wire 115.

Distal coil 140 is slid in a proximal direction over the distal end/tip of the core wire 115 so that the distal end/tip of the components are flush with one another. Adhesive coating 130b is applied to the outer surface of the distal coil 140 before the distal electrical insulation covering 145 (e.g., heat shrink PET) is slid onto the distal end 115b of the core wire 115 covering the entire distal coil 140 from its proximal end 140a to its opposite distal end 140b. Preferably, a proximal end 145a of the distal electrical insulation covering 145 extends in an axial/longitudinal direction proximally of the proximal end 140a of the distal coil 140, while a distal end 145b of the distal electrical insulation covering 145 extends in the axial/longitudinal direction distally of the distal end 140b of the distal coil 140 forming a blunt or atraumatic distal end/tip. When melted, the distal electrical insulation covering 145 (e.g., heat shrink PET) and adhesive coating 130b seeps between the individual winds/loops/turns of the distal coil 140 thereby increasing the bond surface and hence bond strength between the distal electrical insulation covering 145 (e.g., heat shrink PET) and the coil wire 115.

As an assembly, the mechanical securing device (e.g., braid or marker band) 155 is slid on to the distal end 110 until positioned about the distal electrical insulation covering 145 (e.g., heat shrink PET) proximally of the distal coil 140. Without damaging the distal electrical insulation covering 145 the marker band 155 is then physically deformed (e.g., crimped) securing the distal electrical insulation covering 145 about the outer surface of the core wire 115 creating sufficient bond strength to prevent slippage during navigation of the tortuous pathway of the vessel. When the marker band 155 is being crimped proximal of the distal electrical insulation covering 145, the distal coil 140 creates a mechanical lock to increase the separation strength between marker band 155 and distal electrical insulation covering 145. Between the distal end 135b of the glue joint 135 and the proximal end 145a of the distal electrical insulation covering 145 the detachment zone or region of the core wire 115 remains exposed, i.e., free from electrical insulation covering 125, 145.

Following assembly of the electrolytically detachable intravascular delivery device 100, the implantable medical device (e.g., the Medusa Multi-Coil® manufactured by Endoshape, Inc.) 165 is loaded on the distal end 110, as shown in Figures IG-1J. The distal end 110 of the assembled electrolytically detachable intravascular delivery device 100 is threaded through a mechanical securing device (e.g., braid or marker band) 175 before being inserted into the proximal end of the implantable medical device 165. Next, the mechanical securing device (e.g., braid or marker band) 175 is physically deformed (e.g., crimped) securing the proximal end of the implantable medical device 165 to the outer surface of the distal end 110 of the electrolytically detachable intravascular delivery device 100. The mechanical securing device 175 preferably has an axial/longitudinal length (MSD-length) of approximately 0.035″±approximately 0.005″ and an outer diameter (MSD-outer diameter)≤ approximately 0.0185″. To further ensure that the components are secured together, a glue joint 170 is created securing the proximal end of the implantable medical device 165 to the electrolytically detachable intravascular delivery device 100.

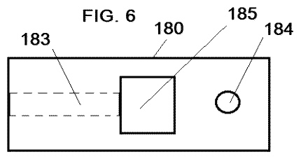

In use, a guidewire is introduced through the vasculature to the target site (e.g., aneurysm) in the body. A catheter is tracked over the guidewire to the target site. Thereafter, the guidewire is withdrawn in a proximal direction from the catheter. Next, the assembled electrolytically detachable intravascular delivery device 100 with the implantable medical device 165 loaded thereon is delivered/navigated through the catheter to the target site. When the implantable medical device is located at the target site, the interventionalist inserts the proximal end 115a of the core/securement/delivery wire 115 into the channel 183 defined in the distal end of the detacher device 180 (FIG. 6). In response to the interventionalist activating a button/switch/dial 184 associated with the detacher device 180 an electrical power source (e.g., battery) and associated electrically circuitry 185 generates an electrical current in the core wire 115. Only the detachment zone or region of the core wire 115 of the electrolytically detachable intravascular delivery device 100 is exposed (free from electrical insulation). Thus, the electrical current transmitted through the core wire 115 concentrates in the exposed detachment zone or region devoid or free of electrical insulation. Within the detachment zone or region, the generated electrical current initiates an electrolysis reaction dissolving the metal core wire 115 thereby releasing/detaching/freeing the implantable medical device 165 from the electrolytically detachable intravascular delivery device 100. Once the core wire 115 is severed along the detachment zone the released implantable medical device 165 remains in place at the target site of the vessel while the electrolytically detachable intravascular delivery device 100 severed/detached therefrom is withdrawn from the catheter in a proximal direction.

The example electrolytically detachable intravascular delivery device is shown and described for using an intrasaccular device for the treatment of an aneurysm. However, the example electrolytically detachable intravascular delivery device may be used to delivery other implantable intravascular medical devices for other treatments.

### Example 1

An electrolytically detachable intravascular delivery system comprising: a core wire made of a nickel-titanium-cobalt alloy, the core wire having a proximal end, an opposite distal end and an outer surface extending therebetween; a distal coil having a proximal end and an opposite distal end with an outer surface extending therebetween, the distal coil disposed about the outer surface of the core wire; a distal adhesive coating on at least a portion of the outer surface of the distal coil; a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil; a proximal coil made of a platinum alloy and disposed about the core wire in the axial direction proximally of the proximal end of the distal coil; the proximal coil having an inner passageway and an outer surface extending in the axial direction from a proximal end to an opposite distal end; a proximal adhesive coating on at least a portion of the outer surface of the proximal coil; a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and a distal glue joint having a distal end and an opposite proximal end abutting in direct physical contact with both the distal end of the proximal coil and the distal end of the proximal electrical insulation covering; and wherein the outer surface of the core wire has an exposed section representing a detachment zone located in the axial direction between the distal end of the distal glue joint and the proximal end of the distal electrical insulation covering.

### Example 2

The system of Example 1, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

### Example 3

The system of any of Examples 1 through 2, further including a spacer coil having a proximal end and an opposite distal end, the spacer coil disposed between the distal most section of the core wire having the smallest outer diameter and an inner surface of the inner passageway of the proximal coil filling a radial gap therebetween.

### Example 4

The system of any of Examples 2 through 3, wherein the proximal end of the proximal coil is substantially aligned with the first transition interface, while the distal end of the proximal coil is substantially aligned with the second transition interface.

### Example 5

The system of any of Examples 1 through 4, further comprising a first mechanical device crimped about the distal electrical insulation covering in the axial direction proximally of the distal coil; the crimped first mechanical device securing the distal electrical insulation covering about the outer surface of the core wire.

### Example 6

The system of any of Examples 1 through 5, the distal end of the distal coil and the distal end of the core wire are flush with one another.

### Example 7

The system of any of Examples 1 through 6, wherein the first and second polyethylene terephthalate materials are identical or different.

### Example 8

An electrolytically detachable intravascular delivery system comprising: a core wire made of a nickel-titanium-cobalt alloy, the core wire having a proximal end, an opposite distal end and an outer surface extending therebetween; a distal coil having a proximal end and an opposite distal end with an outer surface extending therebetween, the distal coil disposed about the outer surface of the coil wire; a distal adhesive coating on at least a portion of the outer surface of the distal coil; a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil; wherein the outer surface of the core wire has an exposed section representing a detachment zone that is located in the axial direction proximally of the proximal end of the distal electrical insulation covering.

### Example 9

An electrolytically detachable intravascular delivery system comprising: a core wire made of a nickel-titanium-cobalt alloy, the core wire having a proximal end, an opposite distal end and an outer surface extending therebetween; a proximal coil made of a platinum alloy and disposed about the outer surface of the core wire; the proximal coil having an inner passageway and an outer surface extending in the axial direction from a proximal end to an opposite distal end; a proximal adhesive coating on at least a portion of the outer surface of the proximal coil; a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and a distal glue joint having a distal end and an opposite proximal end abutting in direct physical contact with both the distal end of the proximal coil and the distal end of the proximal electrical insulation covering; wherein the outer surface of the core wire has an exposed section representing a detachment zone that is located in the axial direction distally of the distal end of the glue joint.

### Example 10

The system of Example 9, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

### Example 11

The system of Example 10, further including a spacer coil having a proximal and an opposite distal end, the spacer coil disposed between the distal most section of the core wire having the smallest outer diameter and an inner surface of the inner passageway of the proximal coil filling a radial gap therebetween.

### Example 12

The system of any of Examples 10 through 11, wherein the proximal end of the proximal coil is substantially aligned with the first transition interface, while the distal end of the proximal coil is substantially aligned with the second transition interface.

### Example 13

An electrolytically detachable intravascular delivery system comprising: a core wire made of a nickel-titanium-cobalt alloy, the core wire having a proximal end, an opposite distal end and an outer surface extending therebetween; a distal coil having a proximal end and an opposite distal end with an outer surface extending therebetween, the distal coil disposed about the outer surface of the core wire; a distal adhesive coating on at least a portion of the outer surface of the distal coil; and a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil; wherein the outer surface of the core wire has an exposed section representing a detachment zone located in the axial direction proximally of the proximal end of the distal electrical insulation covering.

### Example 14

A method of manufacture of an electrolytically detachable intravascular delivery system, the system including: a core wire made of a nickel-titanium-cobalt alloy, the core wire having a proximal end, an opposite distal end and an outer surface extending therebetween; a distal coil having a proximal end and an opposite distal end with an outer surface extending therebetween, the distal coil disposed about the outer surface of the core wire; a distal adhesive coating on at least a portion of the outer surface of the distal coil; a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil; a proximal coil made of a platinum alloy and disposed about the core wire in the axial direction proximally of the proximal end of the distal coil; the proximal coil having an inner passageway and an outer surface extending in the axial direction from a proximal end to an opposite distal end; a proximal adhesive coating on at least a portion of the outer surface of the proximal coil; a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and a distal glue joint having a distal end and an opposite proximal end abutting in direct physical contact with both the distal end of the proximal coil and the distal end of the proximal electrical insulation covering; wherein the outer surface of the core wire has an exposed section representing a detachment zone located in the axial direction between the distal end of the distal glue joint and the proximal end of the distal electrical insulation covering; the method comprising the steps of: threading the core wire through the inner passageway of the proximal coil; applying the proximal adhesive coating on at least a portion of the outer surface of the proximal coil; adhering the proximal electrical insulation covering onto the proximal coil via the proximal adhesive coating; melting the proximal electrical insulation covering so as to seep between individual turns of the proximal coil; creating the distal glue joint securing the distal end of the proximal coil and the distal end of the proximal electrical insulation covering to the outer surface of the core wire; sliding in a proximal direction the distal coil on to the distal end of the core wire; applying the distal adhesive coating to the outer surface of the distal coil; adhering the distal electrical insulation covering onto the distal coil via the distal adhesive coating; and melting the distal electrical insulation covering so as to seep between individual turns of the distal coil.

### Example 15

The method of Example 14, further comprising the step of securing an implantable medical device to the distal end of the core wire.

### Example 16

The method of any of Examples 14 through 15, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

### Example 17

The method of any of Examples 14 through 16, wherein prior to the threading step, further comprising positioning a spacer coil within the inner passageway of the proximal coil; and the threading step comprises sliding together the proximal coil with the spacer coil so that the proximal end of the proximal coil is substantially aligned with the first transition interface, while the distal end of the proximal coil is substantially aligned with the second transition interface; the spacer coil is sized to fill a radial gap between an inner surface of the inner passageway of the proximal coil and the distal most section of the core wire having the smallest outer diameter.

### Example 18

The method of any of Examples 14 through 17, further comprising crimping a first mechanical device about the distal electrical insulation covering proximally of the distal coil; the crimped first mechanical device securing the distal electrical insulation covering about the outer surface of the core wire.

### Example 19

The method of any of Examples 14 through 18, wherein the distal end of the distal coil and the distal end of the core wire are flush with one another.

Thus, while there have been shown, described, and pointed out fundamental novel features of the disclosure as applied to a preferred example thereof, it will be understood that various omissions, substitutions, and changes in the form and details of the systems/devices illustrated, and in their operation, may be made by those skilled in the art without departing from the spirit and scope of the disclosure. For example, it is expressly intended that all combinations of those elements and/or steps that perform substantially the same function, in substantially the same way, to achieve the same results be within the scope of the disclosure. Substitutions of elements from one described example to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. An electrolytically detachable intravascular delivery system comprising:
a core wire made of a nickel-titanium-cobalt alloy, the core wire having a proximal end, an opposite distal end and an outer surface extending therebetween;
a distal coil having a proximal end and an opposite distal end with an outer surface extending therebetween, the distal coil disposed about the outer surface of the core wire;
a distal adhesive coating on at least a portion of the outer surface of the distal coil;
a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil;
a proximal coil made of a platinum alloy and disposed about the core wire in the axial direction proximally of the proximal end of the distal coil; the proximal coil having an inner passageway and an outer surface extending in the axial direction from a proximal end to an opposite distal end;
a proximal adhesive coating on at least a portion of the outer surface of the proximal coil;
a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and
a distal glue joint having a distal end and an opposite proximal end abutting in direct physical contact with both the distal end of the proximal coil and the distal end of the proximal electrical insulation covering; and
wherein the outer surface of the core wire has an exposed section representing a detachment zone located in the axial direction between the distal end of the distal glue joint and the proximal end of the distal electrical insulation covering.

2. The system in accordance with claim 1, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

3. The system in accordance with claim 2, further including a spacer coil having a proximal end and an opposite distal end, the spacer coil disposed between the distal most section of the core wire having the smallest outer diameter and an inner surface of the inner passageway of the proximal coil filling a radial gap therebetween.

4. The system in accordance with claim 3, wherein the proximal end of the proximal coil is substantially aligned with the first transition interface, while the distal end of the proximal coil is substantially aligned with the second transition interface.

5. The system in accordance with claim 1, further comprising a first mechanical device crimped about the distal electrical insulation covering in the axial direction proximally of the distal coil; the crimped first mechanical device securing the distal electrical insulation covering about the outer surface of the core wire.

6. The system in accordance with claim 1, the distal end of the distal coil and the distal end of the core wire are flush with one another.

7. The system in accordance with claim 1, wherein the first and second polyethylene terephthalate materials are identical or different.

8. An electrolytically detachable intravascular delivery system comprising:
a core wire made of a nickel-titanium-cobalt alloy, the core wire having a proximal end, an opposite distal end and an outer surface extending therebetween;
a distal coil having a proximal end and an opposite distal end with an outer surface extending therebetween, the distal coil disposed about the outer surface of the coil wire;
a distal adhesive coating on at least a portion of the outer surface of the distal coil;
a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil;
wherein the outer surface of the core wire has an exposed section representing a detachment zone that is located in the axial direction proximally of the proximal end of the distal electrical insulation covering.

9. An electrolytically detachable intravascular delivery system comprising:
a core wire made of a nickel-titanium-cobalt alloy, the core wire having a proximal end, an opposite distal end and an outer surface extending therebetween;
a proximal coil made of a platinum alloy and disposed about the outer surface of the core wire; the proximal coil having an inner passageway and an outer surface extending in the axial direction from a proximal end to an opposite distal end;
a proximal adhesive coating on at least a portion of the outer surface of the proximal coil;
a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and
a distal glue joint having a distal end and an opposite proximal end abutting in direct physical contact with both the distal end of the proximal coil and the distal end of the proximal electrical insulation covering;
wherein the outer surface of the core wire has an exposed section representing a detachment zone that is located in the axial direction distally of the distal end of the glue joint.

10. The system in accordance with claim 9, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

11. The system in accordance with claim 10, further including a spacer coil having a proximal and an opposite distal end, the spacer coil disposed between the distal most section of the core wire having the smallest outer diameter and an inner surface of the inner passageway of the proximal coil filling a radial gap therebetween.

12. The system in accordance with claim 11, wherein the proximal end of the proximal coil is substantially aligned with the first transition interface, while the distal end of the proximal coil is substantially aligned with the second transition interface.

13. An electrolytically detachable intravascular delivery system comprising:
a core wire made of a nickel-titanium-cobalt alloy, the core wire having a proximal end, an opposite distal end and an outer surface extending therebetween;
a distal coil having a proximal end and an opposite distal end with an outer surface extending therebetween, the distal coil disposed about the outer surface of the core wire;
a distal adhesive coating on at least a portion of the outer surface of the distal coil; and
a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil;
wherein the outer surface of the core wire has an exposed section representing a detachment zone located in the axial direction proximally of the proximal end of the distal electrical insulation covering.

14. A method of manufacture of an electrolytically detachable intravascular delivery system, the system including: a core wire made of a nickel-titanium-cobalt alloy, the core wire having a proximal end, an opposite distal end and an outer surface extending therebetween; a distal coil having a proximal end and an opposite distal end with an outer surface extending therebetween, the distal coil disposed about the outer surface of the core wire; a distal adhesive coating on at least a portion of the outer surface of the distal coil; a distal electrical insulation covering made of a first polyethylene terephthalate material adhered to the outer surface of the distal coil via the distal adhesive coating disposed therebetween; the distal electrical insulation covering having a proximal end and an opposite distal end; the proximal end of the distal electrical insulation covering extending in the axial direction proximally of the proximal end of the distal coil and the distal end of the distal electrical insulation covering extending in the axial direction distally of the distal end of the distal coil; a proximal coil made of a platinum alloy and disposed about the core wire in the axial direction proximally of the proximal end of the distal coil; the proximal coil having an inner passageway and an outer surface extending in the axial direction from a proximal end to an opposite distal end; a proximal adhesive coating on at least a portion of the outer surface of the proximal coil; a proximal electrical insulation covering made of a second polyethylene terephthalate material adhered to the outer surface of the proximal coil via the proximal adhesive coating disposed therebetween; the proximal electrical insulation covering having a proximal end and an opposite distal end; the proximal end of the proximal electrical insulation covering extending in the axial direction proximally of the proximal end of the proximal coil and the distal end of the proximal electrical insulation covering being flush with the distal end of the proximal coil; and a distal glue joint having a distal end and an opposite proximal end abutting in direct physical contact with both the distal end of the proximal coil and the distal end of the proximal electrical insulation covering;
wherein the outer surface of the core wire has an exposed section representing a detachment zone located in the axial direction between the distal end of the distal glue joint and the proximal end of the distal electrical insulation covering; the method comprising the steps of:
threading the core wire through the inner passageway of the proximal coil;
applying the proximal adhesive coating on at least a portion of the outer surface of the proximal coil;
adhering the proximal electrical insulation covering onto the proximal coil via the proximal adhesive coating;
melting the proximal electrical insulation covering so as to seep between individual turns of the proximal coil;
creating the distal glue joint securing the distal end of the proximal coil and the distal end of the proximal electrical insulation covering to the outer surface of the core wire;
sliding in a proximal direction the distal coil on to the distal end of the core wire;
applying the distal adhesive coating to the outer surface of the distal coil;
adhering the distal electrical insulation covering onto the distal coil via the distal adhesive coating; and
melting the distal electrical insulation covering so as to seep between individual turns of the distal coil.

15. The method in accordance with claim 14, further comprising the step of securing an implantable medical device to the distal end of the core wire.

16. The method in accordance with claim 14, wherein the core wire has a plurality of transitions of reductions in outer diameter so that a first outer diameter at the distal end of the core wire is smaller than a second outer diameter at the proximal end of the core wire; wherein the plurality of transitions of reductions in outer diameter includes a first transition interface representing a largest reduction in outer diameter of the core wire and a second transition interface representing a distal most section of the core wire having a smallest outer diameter.

17. The method in accordance with claim 16, wherein prior to the threading step, further comprising positioning a spacer coil within the inner passageway of the proximal coil; and the threading step comprises sliding together the proximal coil with the spacer coil so that the proximal end of the proximal coil is substantially aligned with the first transition interface, while the distal end of the proximal coil is substantially aligned with the second transition interface; the spacer coil is sized to fill a radial gap between an inner surface of the inner passageway of the proximal coil and the distal most section of the core wire having the smallest outer diameter.

18. The method in accordance with claim 14, further comprising crimping a first mechanical device about the distal electrical insulation covering proximally of the distal coil; the crimped first mechanical device securing the distal electrical insulation covering about the outer surface of the core wire.

19. The method in accordance with claim 14, wherein the distal end of the distal coil and the distal end of the core wire are flush with one another.
